(19) United States
(12) Patent Application Publication
(10) Pub. No.: US 2016/0106773 A1
(43) Pub. Date: Apr. 21, 2016

(54) TREATMENT FOR DISEASES RELYING ON DISCOVERY THAT THIOREDOXIN MEDIATES NITRIC OXIDE RELEASE IN CELLS

(71) Applicant: Duke University, Durham, NC (US)

(72) Inventors: Jonathan S. STAMLER, Chapel Hill, NC (US); Moran BENHAR, Chapel Hill, NC (US)

(21) Appl. No.: 14/979,904

(22) Filed: Dec. 28, 2015

Related U.S. Application Data
(63) Continuation of application No. 12/991,445, filed on Jan. 11, 2011.
(60) Provisional application No. 61/071,631, filed on May 9, 2008.

(51) Int. Cl. A61K 31/7135; A61K 31/17; A61K 31/198

(52) U.S. Cl. CPC A61K 31/7135; A61K 31/198; A61K 31/17

(57) ABSTRACT

Patients having a disease associated with high level of thioredoxin system activity or a requirement for nitric oxide, e.g. large cell lymphoma or restenosis, are treated with a thioredoxin reductase inhibitor, e.g. auranofin or arsenic trioxide, and a nitric oxide donating compound, e.g. isosorbide mononitrite or isosorbide dinitrite or nitroglycerin or S-nittrosothiol. Patients having a disease associated with nitric oxide synthase overexpression or increased activity, e.g. Parkinson's disease or septic shock or pancreatic cancer, are treated with Trx/Trx reductase upregulator, e.g. aptamer that binds to thioredoxin reductase inhibitor, and agent causing depletion of nitric oxide (or adduct thereof), e.g. L-NMMA or L-NAME or minocycline or ascorbate or N-acetylcysteine.

### CROSS-REFERENCE TO RELATED APPLICATIONS

This application is a continuation of U.S. patent application Ser. No. 14/991,445, filed Jan. 11, 2011, which is a 371 of International Application No. PCT/US2009/002825, filed May 7, 2009, which claims benefit under 35 U.S.C. § 119(e) of U.S. Provisional Application No. 61/071,631, filed May 9, 2008, the entire contents which are incorporated herein by reference.

### FIELD OF THE INVENTION

This invention in one embodiment is directed at treatment of a disease associated with nitric oxide deficiency or a requirement for nitric oxide and in a second embodiment is directed at treatment of a disease associated with nitric oxide synthase overexpression or increased activity.

### BACKGROUND OF THE INVENTION

In stress situations, protein disulfides form in the body interfering with the normal function of the protein. It is known that the thioredoxin system (i.e. thioredoxin/thioredoxin reductase) present in the body reduces the disulfide groups to cysteine thereby restoring the normal function of the protein. As a result of this reduction, the thioredoxin is oxidized and becomes temporarily inactive. It is known that thioredoxin reductase present in the body causes reduction of oxidized thioredoxin, thereby restoring its activity and functionality.

Furthermore, it is indicated in published literature that under physiological conditions thioredoxin mediates S-nitrosylation of proteins. See Haendeler et al., Nat. Cell Biol. 4, 743 (10/2002); Mitchell, K.A, et al., Natl. Chem. Biol. 1, 154, (8/2005); and Weichsel, A., et al., Biochemistry 46, 1219 (02/06/2007). It is known in the art that NO donors can upregulate thioredoxin and S-nitrosoglutathione reductase (GSNOR).

### SUMMARY OF THE INVENTION

It has been discovered herein that thioredoxin system (hereinafter Trx unless the context indicates otherwise) mediates endogenous nitric oxide release in cells by cysteine containing protein denitrosylation. It follows that Trx reductase inhibitors will cause increase in nitric oxide in cells and that Trx reductase upregulation and Trx inhibitors will cause decrease in nitric oxide in cells.

In one embodiment herein, denoted the first embodiment, the invention herein is directed at treating a patient having a disease associated with a high level of thioredoxin system or a requirement for nitric oxide or a patient benefiting from additional nitric oxide, comprising administering therapeutically effective amounts of a Trx system inhibitor and of a nitric oxide donating compound or other agent that increases nitrosative stress in the patient, (e.g. compound raising endogenous nitric oxide levels).

In another embodiment herein, denoted the second embodiment, the invention herein is directed at a method for treating a patient having a disease associated with nitric oxide synthase overexpression or increased activity, comprising administering to the patient therapeutically effective amounts of Trx system upregulator or activator in the patient and/or of said upregulator or activator of thioredoxin system in the patient and of an agent causing depletion of nitric oxide in the patient.

In still another embodiment herein, denoted the third embodiment, the invention herein is directed at a composition comprising therapeutically effective amounts of a Trx system inhibitor, especially a thioredoxin reductase inhibitor, and of an agent that increases nitrosative stress in a patient.

As used herein, the term thioredoxin system means thioredoxin together with thioredoxin reductase. Thioredoxin system inhibitors include thioredoxin reductase inhibitors and/or thioredoxin inhibitors.

### DETAILED DESCRIPTION

We turn now to the first embodiment herein.

The method of the first embodiment is directed to treating a patient having a disease associated with low level of Trx system or a requirement for nitric oxide or a benefit from additional nitric oxide. Determination of whether or not a disease is associated with a low level of thioredoxin system can be carried out by biopsy and measuring expression by Western or Northern blotting or activity assessment. Determination of whether or not a disease is associated with a requirement for nitric oxide or a patient benefiting from nitric oxide can be carried out by measuring nitric oxide in blood or tissue and comparing to normal or by testing the effect of NO donors on cells. Examples of diseases treatable in the first embodiment herein are apoptosis, atherosclerosis, cancers associated with a low level of Trx or a requirement for nitric oxide, sickle cell disease, heart failure, impotence, asthma, cystic fibrosis, pulmonary hypertension, restenosis and infection, e.g. a bacterial, fungal or viral infection, e.g. tuberculosis or methacillin-resistant Staphylococcus aureus infection. Cancers associated with a low level of Trx or a requirement for nitric oxide include, for example, large cell lymphoma, prostate cancer and lung cancer.

We turn now to the Trx system inhibitors.

Whether or not a compound is a Trx system inhibitor especially a Trx reductase inhibitor can be determined by a standard assay for Trx reductase functionality, e.g. using a Trx reductase assay kit available from Signa-Aldrich, St. Louis, Missouri or by an in vitro assay involving thioredoxin, thioredoxin reductase, S-nitrosylated protein and potential inhibitor. Examples of Trx reductase inhibitors include arsenicals, e.g. arsenic trioxide, gold compounds, e.g. auranofin, antisense to Trx system, aptamer to thioredoxin, siRNA that interferes with Trx reductase or thioredoxin expression and shRNA that silences Trx reductase or thioredoxin expression.

A therapeutically effective amount of Trx reductase inhibitor is an amount that causes amelioration of symptoms and/or pathology of the disease being treated in the first embodiment herein. For auranofin, an oral dose is 6 mg once a day or 3 mg twice a day and an injection dose is 20-55 mg once a week.

Whether or not a compound is an inhibitor of Trx can be determined by the Trx/Trx reductase assay discussed above. Examples of Trx inhibitors are mercurials (e.g. thimerosal or methyl mercury), and thioredoxin-interacting protein (TrxIP).

A therapeutically effective amount of inhibitor of Trx is an amount that causes amelioration of symptoms and/or pathology of the disease being treated in the second embodiment herein. Dosage and routes of administration for thimerosal is 50 μg IV and for methyl mercury is less than 50 μg/liter of blood accumulated dose. Dosage for TrxIP is dose supplied by gene therapy, and if necessary can be assayed by change in SNO level or cellular Trx activity or change in Trx -related function.

We turn now to the nitric oxide donating compound treating agents for the first embodiment.

The nitric oxide donating compounds are compounds capable of transferring NO′, NO⁺, NO′ or NO₂⁺ to cysteine of proteins in the body. A test for determining whether a compound is a nitric oxide donating compound is carried out by determining dilation of blood vessels in an organ chamber bioassay or by determining activation of guanylate cyclase. The nitric oxide donating compound is, for example, a nitrate or a nitrite and can be selected, for example, from the group consisting of inorganic nitrite, isosorbide mononitrate, isosorbide dinitrate, ethyl nitrite, amyl nitrite, nitroglycerin, nitrosothiols and nitroprussides. Nitrosothiols include S-nitrosothiols, for example, S-nitrosoglutathione, S-nitroso-N-acetylpenicillamine, S-nitrosocysteine, S-nitroso-gamma-methyl-L-homocysteine, S-nitroso-L-homocysteine, S-nitroso-gamma-thio-L-leucine and S-nitroso-δ-thio-L-leucine and combinations thereof.

An effective amount of nitric oxide donating compound is an amount that causes amelioration of symptoms or pathology of the disease being treated in the first embodiment herein. When the nitric oxide donating compound is FDA approved, the FDA approved dosage is used if it causes the amelioration described above. For isosorbide mononitrate, a suitable dosage ranges from, for example, 5 mg to 250 mg. For isosorbide dinitrate, a suitable dosage ranges, for example, from 5 mg to 250 mg. For nitrosothiols the dosage ranges from 1 μg to 10 gm/kg and often 10 μg to 1 gm/kg or 10 μg to 100 mg/kg body weight per day.

Routes of administration for the nitrates and nitrites can be, for example, sublingual, topical, intravenous or oral. For isosorbide mononitrate, a preferred route of administration is oral. For isosorbide dinitrate, a preferred route for administration is oral.

Other agents that increase nitrosative stress and increase endogenous notric oxide levels are denitrosylase inhibitors, e.g. the glutathione dependent denitrosylase inhibitor BCNU (carmustine) and glutathione synthesis inhibitors as described in the discretion of the third embodiment e.g. L-buthionine-SR-sulfoximine and the therapeutically effective dosages as described in the description of the third embodiment.

We turn now to the second embodiment herein.

The method of the second embodiment is directed to treating a patient having a disease associated with nitric oxide synthase overexpression or increased activity (i.e. increased compared to normal).

Whether or not a disease is associated with nitric oxide synthase overexpression or increased nitric oxide synthase activity can be determined by presence of increased levels of nitric oxide in blood or increased level of S-nitrosylation of proteins in tissue. Examples of diseases treatable in the second embodiment herein are Alzheimer's disease, Parkinson's disease, congenital diseases of the skeletal muscle (e.g. malignant hyperthermia and muscular dystrophy), muscle weakness/fatigue diabetes, septic shock, cancers associated with nitric oxide synthase overexpression or increased activity (e.g. pancreatic cancer), ulcerative colitis, arthritis and adult respiratory distress syndrome (ARDS).

We turn now to the treating agents for the second embodiment.

Whether or not a compound is a Trx/Trx reductase (TrxR) upregulator can be determined by determining whether it causes increased expression in cells as determined by Western blotting or whether thioredoxin interacting protein (TrxIP) is down or whether there is increased activity of Trx in the tissue.

Examples of Trx reductase upregulators are gene therapy agent for Trx reductase expression, inhibitor of protein that inhibits Trx reductase inhibitor, antisense to Trx reductase inhibitor and aptamer that binds to Trx reductase inhibitor protein. An aptamer for treatment Parkinson's disease is aptamer directed at Parkin. An aptamer for treating malignant hypothermia is aptamer to ryanodine receptor. An aptamer for treatment of diabetes is aptamer to GSK beta.

A therapeutically effective amount of Trx reductase upregulator is an amount that causes amelioration of symptoms and/or pathology of the disease being treated in the first embodiment herein.

Whether or not a compound causes depletion of nitric oxide in the body can be determined by measuring nitric oxide level in blood or by beneficial activity of cells or tissue.

Examples of agents causing depletion of nitric oxide in the body are nitric oxide synthase inhibitors and denitrosylating agents (agents that remove NO groups from protein or from cysteine residues), e.g., ascorbate, e.g. sodium ascorbate (which also upregulates Trx/TrxR) or potassium ascorbate, thiosulfate, hydrogen sulfide (gas or salt), cysteine and N-acetylcysteine (NAC) or other thiols, and deprenyl (prevents nitrosylation of proteins). The denitrosylating agents may often also raise the concentration of Trx reductase in blood or tissue.

Whether or not a compound is a nitric oxide synthase inhibitor can be determined by an assay effective in vitro. Examples of nitric oxide synthase inhibitors useful herein are Ng-monomethyl-L-arginine, monoacetate salt (L-NMMA), N^{g}-nitro-L-arginine, methylester, hydrochloride salt (L-NAME), minocycline, tetracycline, deprenyl, cavtratin, and dexamethasone.

A therapeutically effective amount of compound that causes depletion of nitric oxide is the one that causes amelioration of symptoms and/or pathology of the disease being treated in the second embodiment herein. Therapeutically effective amounts of L-NMMA range from 1 mg/kg to 20 mg/kg and preferred route of administration is intravenous or oral. Therapeutically effective amount of L-NAME ranges from 1 to 100 mg/gm of body weight and preferred route of administration is intravenous. Therapeutically effective amount of dexamethasone range from 1 mg/kg to 2 mg/kg and preferred route of administration is oral. Therapeutically effective amount of ascorbate ranges from 0.25 to 5 mg/g of body weight and preferred route administration is oral or intravenous. Therapeutically effective amounts of NaHS and cysteine are final concentration of 10 μM-mM in blood. Therapeutically effective amount for tetracycline is 100 mg to 2 gm/day. Therapeutically effective amount of N-acetylcysteine is, for example, 300 mg three times a day by mouth. Therapeutically effective amount of deprenyl is 10-15 mg/day orally.

It has been further discovered that patients with a disease associated with nitric overexpression or increased activity is advantageously treated with low dose NO donor (e.g. final concentration of 0.1 nM to 500 nM NO donor plus deprenyl (10-15 mg/day) with or without ascorbate (0.25 to 5 mg/g of body weight) as the low dose NO donor upregulates Trx reductase and also GSNOR.

We turn now to the third embodiment herein.

Disorders treated are these treated for the first embodiment.

In one case of the third embodiment the agent that increases nitrosative stress is a nitric oxide donating compound.

Trx system inhibitors for this case are those discussed above for the first embodiment and nitric oxide donating compounds for the third embodiment are those discussed above for the first embodiment. Therapeutically effective amounts are those discussed above for the first embodiment. A preferred composition comprises auranofin and isosorbide mononitrite; a single dosage form preferably comprises from 1 to 10 mg auranofin and from 10 to 200 mg isosorbide mononitrite, e.g. as a capsule, administered orally.

In a second case of the third embodiment, the agent that causes increase in nitrosative stress is an inhibitor of a denitrosylase e.g. BCNU (carmustine), which inhibits the glutathione dependent denitrosylase GSNOR and thereby increases nitrosative stress.

Trx system inhibitors for this case are these discussed above for the first embodiment and therapeutically effective amounts are those discussed above for the first embodiment. Therapeutically effective amounts of BCNU are 150-200 mg/m² every 4-6 weeks. A preferred composition comprises auranofin and BCNU. A single dosage form preferably comprises 1 to 10 mg auranofin and 150-200 mg/m² BCNU, e.g. as a capsule administered orally. The combination of auranofin and BCNU can also be administered IV (dosage auranofin 3 mg BID; dosage BCNU (150-200 mg/m²))

In a third case of the third embodiment, the agent that causes increase in nitrosative stress is a glutathione synthesis inhibitor e.g. a gamma glutamyl transpeptidase inhibitor (e.g. acivin or the non-glutamine analogue O U 749) or gamma-glutamylcysteine synthetase inhibitor (e.g. buthionine sulfoximine (BSO). In this case the Trx system inhibitors and therapeutically effective doasage thereof are the same as for the first embodiment. A preferred glutathione synthesis inhibitor is L-buthionine-SR-sulfoximine (L-BSO) e.g. at an oral dose of 5-8 gm every 6 hours. A single dosage form preferably comprises 0.25 to 2.5 mg auranofin and 5-8 gm L-BSO administered every 6 hours, e.g. as a capsule orally. The combination of L-BSO and auranofin can also be administered together IV (0.75 g/m² per hr L-BSO and dosage 3 mg BID auraofin per hour)

Elements of the invention and background examples showing thioredoxins denitrosylate cytosolic caspase-3 are set forth in Behar, M. et al, “ Regulated Protein Demtrosylalation by Cystosolic and Mitochondrial Thioredoxins”, Science 320, 1050-1054 (23 May 2008) and Supporting Online Material therefor published 23 May 2008, the whole of which are incorporated herein by reference.

The following working examples illustrate the invention.

### Working Example I

A 50 year old with large cell lymphoma disease who was not responding to treatment was given by mouth 6 mg auranofin and 50 mg isosorbide dinitrate once a day for a month. Tumor burden decreases as determined by CAT scan.

### Working Example II

A 60 year old with recurrent restenosis is give 0.15 mg/kg/day IV arsenic trioxide and either 60 mg isosorbide mononitrate per day or nitroglycerin patch of 1-2 inches up to 4 times a day. After one month disease abates and incidence of thrombotic complication disappears and restenosis does not reoccur.

### Working Example III

A 70 year old with Parkinson's disease is given orally 300 mg N-acetylcysteine and 0.3 mg dose by injection into spinal fluid (range may be 10 μg-2 mg of body weight) of aptamer directed at Parkin for 30 days. Movement improves. Ascorbate 1 gm/day is them added and dexterity improves further.

### Working Example IV

A 75 year old with Alzheimer's is then started on combination of NAC (300 mg/day), ascorbate 1 gm/day and minocycline 100 mg TID and memory improves.

### Working Example V

A 13 year old with muscular dystrophy is given deprenyl 10 mg/day plus isosorbide dinitrate (ISDN) 10 mg/day and weakness improves. Minocycline 300 mg BID is added with further improvement in 6 minute walking test.

### Working Example VI

A 65 year with Alzheimers is administered deprenyl 15 mg and S-nitrosoglutathione 10 mg po TID. Blood pressure is unaltered but memory improves.

### Working Example VII

A 40 year old with rhematoid arthritis is administered deprenyl 10 mg plus ascorbate 1 gm plus ISDN 5 mg PO TID or plus cysteine or NaHS (final concentration in blood of 10 μM-1 mM). Progression of the disease slows.

### Working Example VIII

A 40 year old with central core disease undergoes operation for pancreatic cancer and course complicated by malignant hyperthermia (MH). The patient is administered deprenyl 10 mg and ISDN 5 mg po tid with acute relief of MH and improvement in muscle function over time.

### Working Example IX

A 40 year old with septic shock is given gene therapy for TrxIP and 15 mg/kg intravenous L-NMMA (2 mg/kg IV TID). Blood pressure increases and stabilizes.

### Working Example X

A 40 year old with pancreatic cancer is given gene therapy for TrxIP and by infusion per day 10 gms of sodium ascorbate. The cancer regresses.

### Working Example XI

A 70 year old with glioblastoma is given orally a capsule containing 6 mg auranofin and 50 mg isosorbide dinitrate or 6 mg auranofin and 150 mg/m² BCNU or 2.5 mg auranofin and 2 gm L-BSO given every 6 hours. Regression in the cancer occurs.

### Variations

The foregoing description of the invention has been presented describing certain operable and preferred embodiments. It is not intended that the invention should be so limited since variations and modifications thereof will be obvious to those skilled in the art, all of which are within the spirit and scope of the invention.

1. A method for treating a patient having cancer, comprising administering therapeutically effective amounts of (i) a thioredoxin system inhibitor and of (ii) an agent that causes an increase in nitrosative stress to a patient in need thereof.

2. The method of claim 1, wherein the cancer is selected from the group consisting of large cell lymphoma, prostate cancer and lung cancer.

3. The method of claim 2, wherein the cancer is large cell lymphoma.

4. The method of claim 2, wherein the cancer is prostate cancer.

5. The method of claim 2, wherein the cancer is lung cancer.

6. The method of claim 1, wherein the thioredoxin system inhibitor is selected from the group consisting of mercurials, arsenicals, gold compounds, thioredoxin interacting protein, antisense to thioredoxin system, aptamers to thioredoxin, siRNA that interferes with thioredoxin system reductase or thioredoxin expression, and shRNA that silences thioredoxin reductase expression.

7. The method of claim 6, wherein the thioredoxin system inhibitor is a gold compound.

8. The method of claim 7, wherein the gold compound is auranofin.

9. The method of claim 1, wherein the agent that causes an increase in nitrosative stress is a glutathione synthesis inhibitor.

10. The method of claim 9, wherein the glutathione synthesis inhibitor is selected from the group consisting of gamma glutamyl transpeptidase inhibitors and gamma-glutamylcysteine synthetase inhibitors.

11. The method of claim 10, wherein the glutathione synthesis inhibitor is a gamma glutamyl transpeptidase inhibitor.

12. The method of claim 11, wherein the gamma glutamyl transpeptidase inhibitor is selected from the group consisting of acivin and the non-glutamine analogue OU749.

13. The method of claim 10, wherein the glutathione synthesis inhibitor is a gamma-glutamylcysteine synthetase inhibitor.

14. The method of claim 13, wherein the gamma-glutamylcysteine synthetase inhibitor is selected from the group consisting of buthionine sulfoximine (BSO) and L-buthionine-SR-sulfoximine (L-BSO).

15. The method of claim 13, wherein the gamma-glutamylcysteine synthetase inhibitor is L-BSO.

16. A method for treating a patient having cancer, comprising administering therapeutically effective amounts of (i) auranofin and of (ii) L-BSO.

17. The method of claim 16, wherein the auranofin is administered orally at a dose of 0.25 to 2.5 mg every six hours.

18. The method of claim 16, wherein the L-BSO is administered orally at a dose of 5-8 gm every six hours.

19. The method of claim 16, wherein the auranofin is administered intravenously at a dose of 3 mg oral BID.

20. The method of claim 16, wherein the L-BSO is administered intravenously at 0.75 g/m² per hour.

21. The method of claim 14, wherein the gamma-glutamylcysteine synthetase inhibitor is BSO.

22. The method of claim 1, wherein the agent that causes an increase in nitrosative stress is an inhibitor of a denitrosylase.

23. The method of claim 22, wherein the inhibitor of a denitrosylase is BCNU (carmustine).
